(19) United States
(12) Patent Application Publication
(10) Pub. No.: US 2017/0198248 A1
(43) Pub. Date: Jul. 13, 2017

(54) CELL SEPARATION FILTER AND CELL CULTURE VESSEL

(71) Applicant: OPTNICS PRECISION CO., LTD., Ashikaga-shi, Tochigi (JP)

(72) Inventors: Seichin KINUTA, Tochigi (JP); Masashi KOBAYASHI, Tochigi (JP)

(21) Appl. No.: 15/318,671

(22) PCT Filed: Aug. 3, 2016
(86) PCT No.: PCT/JP2016/072863

§ 371 Date: Dec. 14, 2016

(30) Foreign Application Priority Data
Aug. 6, 2015 (JP) 2015-155937

(51) Int. Cl. C12M 1/26; C12M 1/00

(52) U.S. Cl. CPC C12M 33/14; C12M 23/38

(57) ABSTRACT

A cell separation filter has a plate-shaped base portion, one or more porous areas, provided on the base portion, in which holes are formed in order to separate cells for separation from a fluid sample, and one or more wall portions, formed integrally with the base portion, and surrounding the one or more porous areas.

### TECHNICAL FIELD

The present disclosure relates to a cell separation filter and a cell culture vessel.

### BACKGROUND ART

In Japanese Patent Application Laid-Open (JP-A) No. 2013-541958, a cell harvesting device that includes a screen filter that is used to separate and harvest target cells from a fluid sample such as blood or a physiological fluid is disclosed. In this cell harvesting device, target cells that are contained in the fluid sample are filtered out from non-target cells and harvested. Moreover, a process of selecting cancer cells as the target cells, and selecting red blood cells and white blood cells as the non-target cells is also disclosed.

### SUMMARY OF THE INVENTION

### Technical Problem

Although this is not mentioned in the aforementioned conventional example, the cells that have been separated from the fluid sample are subsequently transferred to an observation instrument or a cultivation vessel and the like and are then utilized.

However, because there are instances in which the cells are damaged during such transfers, it is desirable that the separated cells are touched as little as possible.

It is an object of the present disclosure to make it possible for cells that have been separated from a fluid sample to be used just as they are without having to be transferred to another instrument.

### Solution to the Problem

A cell separation filter according to a first aspect has a plate-shaped base portion, one or more porous areas, provided on the base portion, in which holes are formed in order to separate cells for separation from a fluid sample, and one or more wall portions, formed integrally with the base portion, and surrounding the one or more porous areas.

A fluid sample that contains the cells that are to be separated is supplied from the wall portion side of the cell separation filter towards the opposite side from the wall portions. Cells that are too large to pass through the holes in the porous areas are captured, and are separated from the cells that have passed through the holes. Because the one or more wall portions surrounding the one or more porous areas are formed integrally with the plate-shaped base portion of the cell separation filter, it is easy for the cells that have been captured on the inner side of the one or more wall portions to be trapped on the inner side of the wall portions. Because of this, the cells that have been separated out from the fluid sample can be used just as they are without having to be transferred to another instrument.

A second aspect is the cell separation filter according to the first aspect wherein a plurality of porous areas, each surrounded by the one of a plurality of wall portions, are provided on the base portion.

In this cell separation filter, because the plurality of porous areas, each surrounded by one of the plurality of wall portions, are provided on the base portion, when a fluid sample is passed through the cell separation filter, the cells that are to be separated are captured in each one of the plurality of porous areas. Because the plurality of porous areas are each surrounded by the one of the plurality of wall portions, the cells are able to be used in a plurality of conditions.

A third aspect is the cell separation filter according to the first or second aspects wherein the cell separation filter is formed from metal.

Because this cell separation filter is formed from metal, a reduction in costs can be achieved via improved reusability and the like.

A fourth aspect is the cell separation filter according to the first or second aspects wherein the cell separation filter is formed from resin.

Because this cell separation filter is formed from resin, it is possible to achieve an even greater cost reduction.

A fifth aspect is the cell separation filter according to the fourth aspect wherein the resin is transparent.

Because this cell separation filter is formed from transparent resin, by shining a light from the underside of the cell separation filter, observation of the cells using a microscope can be performed with ease.

A cell cultivation vessel according to a sixth aspect has the cell separation filter according to any one of the first through fifth aspects, and a blocking member that is attached to a surface of the base portion of the cell separation filter which is on an opposite side from the one or more wall portions, and that blocks off the one or more porous areas.

In this cell culture vessel, after the cells have been separated using the cell separation filter, the blocking member is attached to the surface of the base portion that is on the opposite side from the one or more wall portions, and the one or more porous areas are blocked off by this blocking member. As a consequence, a culture liquid can be held on the inner side of the wall portions. As a result, the cells that have been separated out from the fluid sample can be cultured just as they are without having to be transferred to another vessel.

### Advantageous Effects of the Invention

According to the cell separation filter and cell culture vessel according to the present disclosure, the excellent effect is achieved that it is possible to utilize cells that have been separated out from a fluid sample just as they are without having to transfer them to another instrument.

### BRIEF DESCRIPTION OF THE DRAWINGS

FIG. 1 is a perspective view showing a cell separation filter according to the present exemplary embodiment.

FIGS. 2(A) through 2(C) are enlarged cross-sectional views showing various types of hole shape in a porous area, and cells that are captured in the holes.

FIG. 3 is a perspective view showing a variant example of a cell separation filter according to the present exemplary embodiment.

FIG. 4 is a cross-sectional view schematically showing a cell culture vessel according to the present exemplary embodiment.

FIG. 5 is a cross-sectional view schematically showing a process for manufacturing a metal cell separation filter.

FIG. 6 is a cross-sectional view schematically showing a process for manufacturing a resin cell separation filter.

### DESCRIPTION OF THE EMBODIMENTS

Hereinafter, an exemplary embodiment for implementing the present invention will be described based on the drawings.

[Cell Separation Filter]

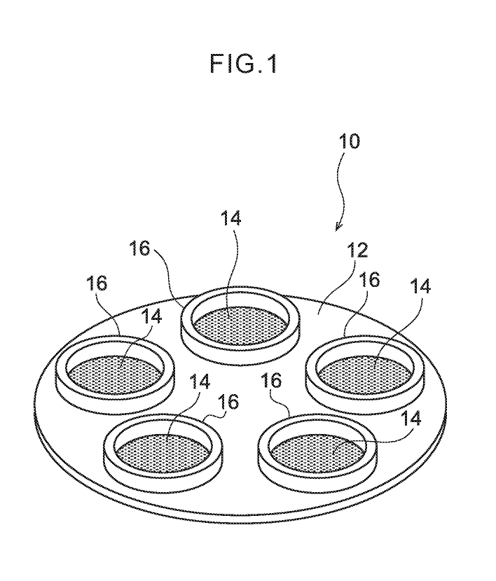

In FIG. 1, a cell separation filter 10 according to the present exemplary embodiment has a plate-shaped base portion 12, porous areas 14, and wall portions 16. This cell separation filter 10 is formed from metal or resin. A cell separation filter 10 that is formed from metal is manufactured, for example, by means of lithography utilizing X-rays or ultraviolet rays or the like, and by electroforming. A cell separation filter 10 that is formed from resin is molded using a metal mold that has been manufactured, for example, by means of lithography utilizing X-rays or ultraviolet rays or the like, and by electroforming. The resin used is preferably transparent, however, an opaque resin may also be used.

The metal material that is used may include, for example, at least one of palladium (Pd), platinum (Pt), gold (Au), silver (Ag), iridium (Ir), rhodium (Rh), and ruthenium (Ru). This material may be a simple metal substance of palladium (Pd), platinum (Pt), gold (Au), silver (Ag), iridium (Ir), rhodium (Rh), or ruthenium (Ru), or may be, for example, an alloy or the like such as a palladium (Pd)/nickel (Ni) alloy, a platinum (Pt)/nickel (Ni) alloy, or a gold (Au)/nickel (Ni) alloy or the like. If an alloy is used, then it is desirable for the proportion of the one of the aforementioned metals that is used to be greater than the proportion of the nickel or the like in the alloy.

Compared with a metal such as, for example, nickel (Ni) or the like, the aforementioned metals have an extremely low toxicity towards cells. The reasons for this are the fact that the toxicity of the palladium (Pd) itself is low, and the fact that because an alloy of Pd and nickel (Ni) is formed as a solid body, it is possible to prevent any elution of the nickel (Ni). Of these, because of the low metal costs and the low toxicity, palladium or a palladium (Pd)/nickel (Ni) alloy are preferable, and, in the case of a Pd/Ni alloy, an alloy in which the Pd is more than 50% (by weight), for example, an alloy of 80% Pd and 20% Ni, is preferable. A Pd and Ni alloy filter, and a Pd filter and the like are acid-resistant and heat-resistant, and can be dyed a variety of colors using a FISH method or the like while in the form of a filter, and can be observed by microscope just as they are (i.e., in an upright state). In addition, they are rigid and extremely durable, and are difficult for cells to adhere to even without undergoing surface processing.

The plate-shaped base portion 12 is formed, for example, in a disk shape. Note that it is sufficient for the base portion 12 to be able to be placed in a filter ring (cassette) that is installed in a filtration unit of a cell separation device (not shown in the drawings), and it is also possible for the base portion 12 to have a square shape. The size of the base potion 12 is suitably determined in consideration of physical factors such as the quantity of a fluid sample such as blood or the like, the diameter of holes 20 (described below), time, flow velocity, and pressure resistance and the like, and in consideration of operability and cost and the like. For example, when 5 mL of blood is being processed, the diameter (in the case of a circular base portion) or the vertical and horizontal dimensions (in the case of a square base portion) are normally between approximately 10˜15 mm, however, the size can be set within, but not limited to, a range of between approximately 5˜20 mm in accordance with the quantity of blood. Moreover, the thickness of the base portion 12 is suitably determined in consideration of the relationship thereof with the hole density, pressure resistance, cost and the like, and is normally set to between 10˜40 μm, and preferably to between approximately 15˜40 μm.

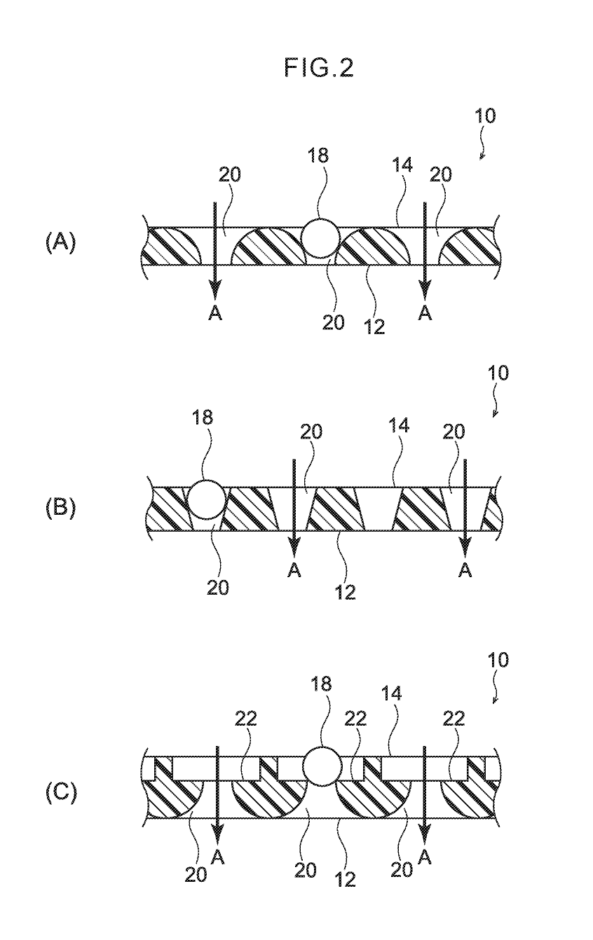

The porous areas 14 are provided on the base portion 12. A large number of holes 20 that are used to separate cells 18 (see FIGS. 2(A)˜2(C)) that are to be separated out from a fluid sample (not shown in the drawings) are formed in the porous areas 14. The holes 20 are arranged uniformly and in a regular pattern. The density of the holes 20 per 1 cm² of the filter surface area differs depending on the type of layout used, however, normally, this density is set to between 1×10⁴˜2×10⁵/cm², and is preferably set to between 5×10⁴˜1×10⁵/cm².

The hole diameter of the holes 20 has a size that is small enough to prevent the cells 18 that are being separated from passing through them, yet large enough to allow cells that are not being separated (not shown in the drawings) to pass through them. The cells 18 that are to be separated are, for example, cancer cells such as peripheral circulating tumor cells (also known as CTC) or rare cells. The size (i.e., the major axis) of the human blood cell components that are not the target of separation was found as a result of histogram analysis to be approximately 6˜7 μm for red blood cells, approximately 7˜9 μm for white blood cells, and less than 5 μm for platelets. In contrast, the size of the cells 18 that are the target of separation is approximately 10˜20 μm. Accordingly, the minimum diameter of the holes 20 is normally approximately 7˜10 μm, and preferably is approximately 7.5˜9 μm, and more preferably is approximately 7.5˜8.5 μm.

The cross-sectional configuration of the holes 20 is set, for example, to the configurations shown in FIG. 2(A)˜FIG. 2(C). In the example shown in FIG. 2(A), the diameter of the holes 20 becomes gradually narrower moving from the entry side (i.e., the upper side) towards the exit side (i.e., the lower side), and the internal wall of the holes 20 is formed having an arc-shaped cross-section that protrudes towards the center side of each hole 20. In the example shown in FIG. 2(B), the holes 20 are formed in a tapered shape in which the diameter becomes gradually narrower moving from the entry side (i.e., the upper side) towards the exit side (i.e., the lower side).

In the example shown in FIG. 2(C), a recess 22 whose diameter is larger than that of the hole 20 is formed at the entry side (i.e., the upper side) of each hole 20. The shape of the entry side (i.e., the lower side) of the holes 20 is formed by vertically inverting the shape of the holes 20 shown in FIG. 2(A). In other words, the diameter of the holes 20 becomes gradually wider moving from the entry side (i.e., the upper side) of each hole 20 towards the exit side (i.e., the lower side) thereof, and the internal wall of the holes 20 is formed having an arc-shaped cross-section that protrudes towards the center side of each hole 20. The size of the recesses 20 should be set to a size that enables the cells 18 that are to be separated to be captured, and this size may be, but is not limited to, a diameter of, for example, 20˜30 μm and a depth of 5˜15 μm, and, preferably, a diameter of 25˜30 μm and a depth of 10 μm.

In FIG. 1, the wall portions 16 are formed integrally with the base portion 12, and surround the porous areas 14. The wall portions 16 are formed, for example, in a toroidal shape, and stand upright on an upper surface side (i.e., on the entry side of the holes 20 in FIG. 2 (A)˜FIG. 2 (C)) of the base portion 12. The height of the wall portions 16 from the upper surface of the base portion 12 is desirably larger than the diameter of the cells 18 that are to be separated, and is, for example, 5˜10 μm, and, preferably, 6˜8 μm.

A plurality of porous areas 14, each surrounded by the one of a plurality of wall portions 16, are provided on the base portion 12. In the example shown in FIG. 1, for example, five porous areas 14 that are surrounded by the wall portions 16 are arranged in a circle. Note that the portions that are not surrounded by the wall portions 16 may also be formed as additional porous areas 14. Moreover, it is also possible to alter the size and shape of the holes 20 in each one of the porous areas 14.

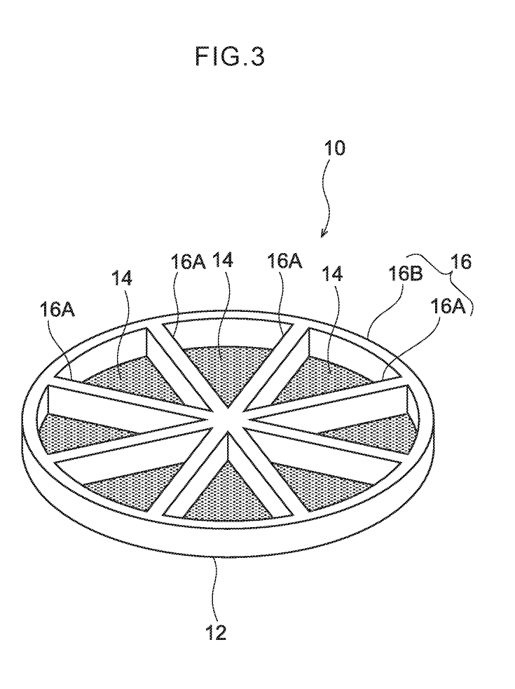

The layout of the wall portions 16 is not limited to the example shown in FIG. 1. As is shown in FIG. 3, it is also possible to form the wall portions 16 from rectilinear portions 16A that extend radially from a central portion of the base portion 12, and a toroidal portion 16B that extends toroidally around an outer circumference of the base portion 12. The rectilinear portions 16A and the toroidal portion 16B are formed integrally so as to surround each one of a plurality of porous areas 14. In this example, eight porous areas 14 that are surrounded by the wall portions 16 are formed in the circumferential direction of the base portion 12. In addition to this, it is also possible to form the wall portions 16 in a lattice configuration.

[Cell Culture Vessel]

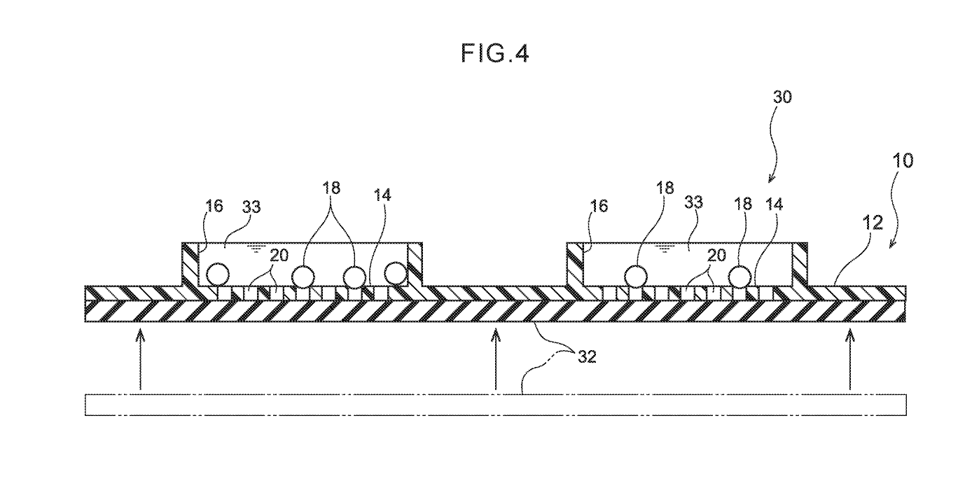

In FIG. 4, a cell culture vessel 30 according to the present exemplary embodiment has a cell separation filter 10 and a blocking member 32. The blocking member 32 is attached to the surface (i.e., the rear surface) of the plate-shaped base portion 12 of the cell separation filter 10 that is on the opposite side from the one or more wall portions 16, and blocks off the one or more porous areas 14. This blocking member 32 is formed, for example, from elastomer or rubber or the like and has the same surface area as the base portion 12. The blocking member 32 is attached by adhesion or bonding to the rear surface side of the cell separation filter 10 once this has captured the cells 18 that are being separated. The holes 20 (see FIG. 2(A)˜FIG. 2 (C) in the porous areas 14 are blocked off by the blocking member 32.

(Action)

The present exemplary embodiment is formed in the above-described manner, and the action thereof is described below. In FIG. 2(A)˜FIG. 2 (C), in the cell separation filter 10 according to the present exemplary embodiment, a fluid sample that contains the cells 18 that are to be separated is supplied from the wall portion 16 side of the cell separation filter in the direction shown by the arrows A towards the opposite side from the wall portions 16. Cells 18 that are too large to pass through the holes 20 in the porous areas 14 are captured, and are separated from the cells (not shown in the drawings) that have passed through the holes 20. Because the wall portions 16 that surround the porous areas 14 are formed integrally with the plate-shaped base portion 12 of the cell separation filter, it is easy to trap the cells 18 that have been captured on the inner side of the wall portions 16 on the inner side of the wall portions 16. Because of this, the cells 18 that have been separated out from the fluid sample can be used just as they are without having to be transferred to another instrument.

In particular, in the present exemplary embodiment, because the plurality of porous areas 14, each surrounded by one of the plurality of wall portions 16, are provided on the base portion 12, when a fluid sample is passed through the cell separation filter 10, the cells 18 that are to be separated (see FIG. 2 (A)˜FIG. 2 (C)) are captured in each one of the plurality of porous areas 14. Because the plurality of porous areas 14 are each surrounded by one of the plurality of the wall portions 16, the cells 18 are able to then be used in a plurality of conditions.

When the cell separation filter 10 is formed from metal, reusability is improved so that a reduction in costs can be achieved. When, however, the cell separation filter is formed from resin, it is possible to achieve an even greater cost reduction compared to a high-cost metal. Furthermore, when the cell separation filter 10 is formed from a transparent resin, then by shining a light from the underside of the cell separation filter 10, observation of the cells 18 using a microscope can be performed with ease. In addition, a cell separation filter 10 that is formed from resin is also disposable.

In FIG. 4, in the cell culture vessel 30, after the cells 18 have been separated using the cell separation filter 10, the blocking member 32 is attached to the surface of the base portion 12 that is on the opposite side from the one or more wall portions 16, and the one or more porous areas 14 are blocked off by this blocking member 32. As a consequence, a culture liquid 33 can be held on the inner side of the wall portions 16. As a result, the cells 18 that have been separated out from the fluid sample can be cultured just as they are without having to be transferred to another vessel. In other words, the cell separation filter 10 can be used as a culture dish. When the plurality of porous areas 14, each surrounded by one of the plurality of wall portions 16, are provided on the base portion 12, the cells 18 that are cultivated in each porous area 14 can be used in conditions that are mutually different from each other. As an example, a plurality of types of anti-cancer drugs can be tested on a single cell culture vessel 30. Moreover, in this case, because it is not necessary to transfer the cells 18 to a plurality of vessels, it is possible to prevent the cells 18 from being damaged during such a transfer and consequently becoming unsuitable for culturing.

[Method of Mass-Producing Metal Cell Separation Filters]

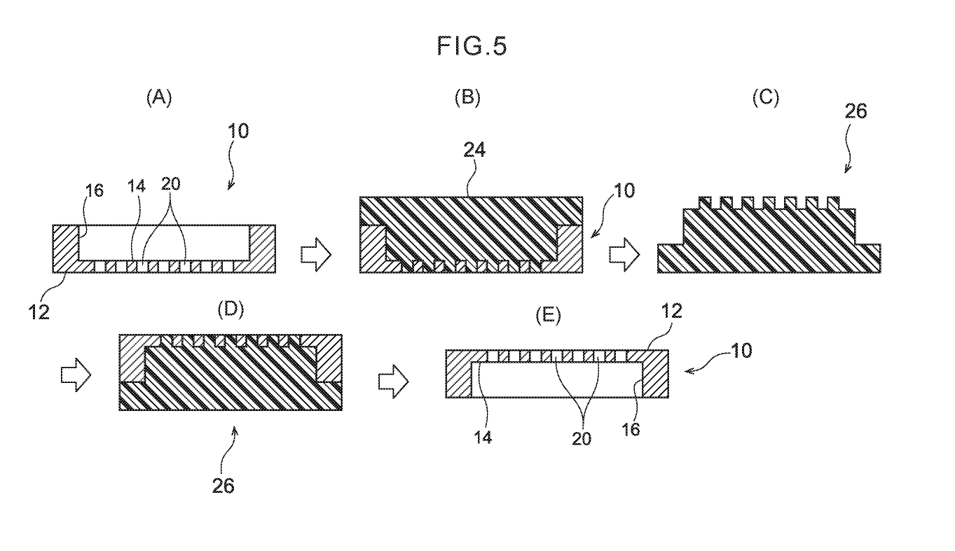

In FIG. 5 (A) through FIG. 5 (E), when metal cell separation filters are being mass-produced, as is described above, a cell separation filter 10 that forms the base is manufactured by means of lithography utilizing X-rays or ultraviolet rays, and by electroforming (FIG. 5 (A)). Next, resin molding is performed on this cell separation filter 10 (FIG. 5 (B)). At this time, care is taken to ensure that resin 24 completely fills the interior of the holes 20 in the porous areas 14. Next, the resin is removed from the mold, so that a resin mold 26 is obtained (FIG. 5 (C)). Electroforming is then performed on this resin mold 26 (FIG. 5 (D)), and the resin mold is then removed from the mold. As a result, a metal cell separation filter 10 can be obtained (FIG. 5 (E)). In the same way, by repeating the electroforming and mold removal steps on the resin mold 26, it is possible to mass-produce metal cell separation filters 10 having the porous areas 14 and the wall portions 16.

[Method of Mass-Producing Resin Cell Separation Filters]

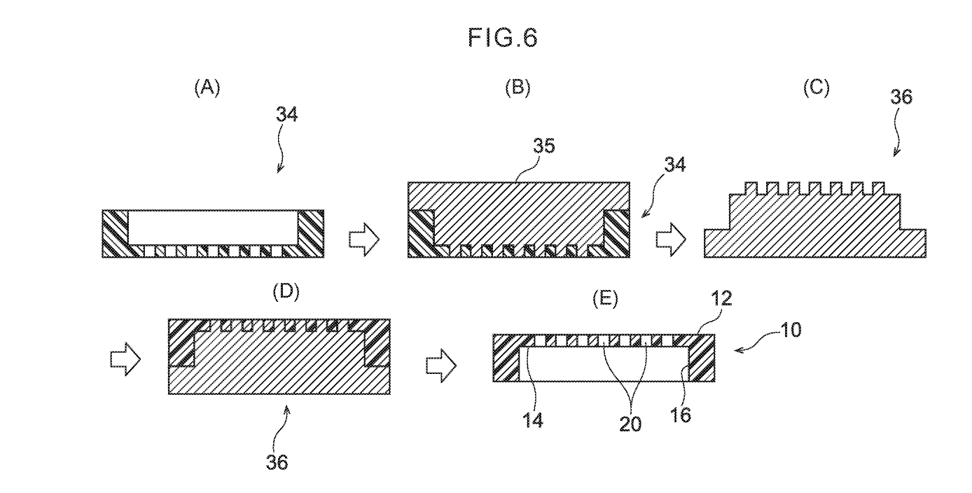

In FIG. 6 (A) through FIG. 6 (E), when resin cell separation filters are being mass-produced, as is described above, a photoresist 34 having a shape that corresponds to the cell separation filter 10 (FIG. 5 (A)) is manufactured by means of lithography utilizing X-rays or ultraviolet rays (FIG. 6 (A)). Next, electroforming is performed on this photoresist 34 (FIG. 6 (B)). At this time, care is taken to ensure that electroforming metal 35 completely fills the interior of the portions of the photoresist 34 that correspond to the holes 20 (FIG. 5 (A)) in the porous areas 14. Next, the photoresist 34 is removed from the mold, so that a metal mold 36 is obtained (FIG. 6 (C)). Resin molding is then performed using this metal mold 36 (FIG. 6 (D)), and the resin mold is then removed from the metal mold. As a result, a resin cell separation filter 10 can be obtained (FIG. 6 (E)). In the same way, by repeating the resin molding and mold removal steps using the metal mold 36, it is possible to mass-produce resin cell separation filters 10 having the porous areas 14 and the wall portions 16.

### ADDITIONAL EXEMPLARY EMBODIMENTS

An example of an exemplary embodiment of the present invention has been described above, however, exemplary embodiments of the present invention are not limited to this and it is to be understood that, in addition to the exemplary embodiment described above, various modifications and the like may be implemented insofar as they do not depart from the spirit or scope of the present invention.

For example, the plurality of porous areas 14, each surrounded by one of the plurality of wall portions 16, are provided on the base portion 12, however, it is also possible for just one porous area 14 that is surrounded by a wall portion 16 to be provided. Moreover, the cell separation filter is formed from metal or resin, however, it may also be formed from some other material.

Priority is claimed on Japanese Patent Application No. 2015-155937, filed Aug. 6, 2015, the disclosure of which is incorporated herein by reference.

All references, patent applications and technical specifications cited in the present specification are incorporated by reference into the present specification to the same extent as if the individual references, patent applications and technical specifications were specifically and individually recited as being incorporated by reference.

1. A cell separation filter comprising:
a plate-shaped base portion;
one or more porous areas, provided on the base portion, in which holes are formed in order to separate cells for separation from a fluid sample; and
one or more wall portions, formed integrally with the base portion, and surrounding the one or more porous areas; wherein
a plurality of porous areas, each surrounded by one of a plurality of wall portions, are provided on the base portion.

2. (canceled)

3. (canceled)

4. (canceled)

5. (canceled)

6. (canceled)

7. A cell culture vessel comprising:
the cell separation filter according to claim 1; and
a blocking member that is attached to a surface of the base portion of the cell separation filter that is on an opposite side from the one or more wall portions, and blocks off the one or more porous areas.

8. A cell culture vessel comprising:
a cell separation filter comprising:
a plate-shaped base portion;
one or more porous areas, provided on the base portion, in which holes are formed in order to separate cells for separation from a fluid sample; and
one or more wall portions, formed integrally with the base portion, and surrounding the one or more porous areas; and
a blocking member that is attached to a surface of the base portion of the cell separation filter that is on an opposite side from the one or more wall portions, and blocks off the one or more porous areas.
